(19) United States
(12) Patent Application Publication
(10) Pub. No.: US 2005/0245933 A1
(43) Pub. Date: Nov. 3, 2005

(54) Multi coaxial screw system

(76) Inventor: Lionel C. Sevrain, West Palm Beach, FL (US)

Correspondence Address: BURNS & LEVINSON LLP, 1030 15TH STREET NW, SUITE 300, WASHINGTON, DC, 20005-1501, US

(21) Appl. No.: 11/115,904

(22) Filed: Apr. 27, 2005

Related U.S. Application Data
(60) Provisional application No. 60/566,942, filed on May 3, 2004.

(51) Int. Cl. A61B 17/58

(52) U.S. Cl. 606/73

(57) ABSTRACT

An anchoring system for bones comprises male and female anchoring members each having proximal and distal ends, the proximal ends being adapted, when the anchoring members are installed in the bone, to be spaced from each other with the male and female anchoring members converging from the proximal ends towards the distal ends. The male and female anchoring members are adapted to be connected to each other in the bone and distally of the proximal ends via a connection mechanism generally in the form of mating threads. The male anchoring member comprises at least first and second sections provided respectively with the thread and with at least part of the connection mechanism, the first and second sections being rotatable independently of one another and being telescopically mounted together.

### PRIORITY

The present invention relates and claims priority under 35 U.S.C 109(e) from U.S. Provisional Application Ser. No. 60/566,942, filed May 3, 2004, entitled “Multi coaxial Screw System”, which is hereby incorporated by reference in its entirety.

### FIELD OF THE INVENTION

The present invention relates to devices for attaching various objects, such as prostheses or implants, to bones, including for anchoring spinal instrumentations to vertebrae of the human rachis and for fixing broken bones.

### BACKGROUND OF THE INVENTION

The use of screw fixation to position bone segments has been established as common practice in the surgical treatment of bone fractures. Threaded fasteners are used to secure various items, such as anchoring plates, to bones. Examples of such threaded fasteners as appear in the prior art are illustrated in FIGS. 1 and 2. Threaded fasteners are generally provided with a single thread configured for the material in which it is to be received such as to provide maximum retention thereof in the material.

A threaded fastener such as a screw typically includes a proximal head adapted to receive a tool for the rotation of the fastener, an intermediate stem provided with a thread that can extend up to or close to the head, and a distal end that is generally sharp to facilitate the piercing of the material (e.g. body tissues) in which the screw must be anchored. The thread is characterised by its pitch (that is, the space between adjacent turns of the thread), its depth (that is, the distance between the edge of the thread and the shank of the fastener), the size of each thread turn (that is, the width of the thread at its bottom, i.e. where it merges with the shank), the shape of the edge of the thread, and the obliqueness of the thread.

The choice of these parameters define a unique thread which has an effect on the solidity if the anchoring of the fastener in a given material, and in the speed of penetration of the fastener in the material.

Therefore, a threaded fastener having different pitches would have two different speeds of penetration. A fastener having two threads of different pitches would result in the breakage of the material, which is of the thread tapped therein by the fastener as a result of the two different speeds of penetration.

In the context of a composite medium, that is, a medium having at least two different materials, a single thread of uniform characteristics often does not provide proper anchoring of the threaded fastener in both materials. Such materials may differ at the level of their texture, density or physical characteristics. In such a case, a threaded fastener having a thread of uniform characteristics will generally not produce an optimal anchoring thereof in view of the different mechanical requirements of each of the materials into which the threaded fastener is inserted. The threaded fastener will be properly anchored in the material for which the thread of the fastener is adequate, but the same thread most likely will not provide adequate anchoring of the fastener in the other material as the thread of the threaded fastener is not well adapted to such other material. As a consequence, there will be loosening, pull-out or breakage of the fastener, or breakage in the materials.

To try to prevent the screws from loosening, various systems have been used, such as directing the screws along different orientations (e.g., diverging or converging); providing a locking mechanism on the screw (e.g., counter-nut); modifying the screw's thread (height and depth); and engaging each screw to two tissues having different densities.

PCT Publication Number WO 01/89400-A2, published on Nov. 29, 2001 in the name of Sevrain, discloses, as shown in FIGS. 1 and 2, discloses an anchoring system S adapted to mount a support plate P/P′ to a pair of adjacent vertebrae V₁ and V₂ of the human rachis. The anchoring system S includes first and second fasteners 10 and 20 each having proximal and distal ends. The proximal ends of each of the threaded fasteners 10 and 20 are adapted to be spaced from each other for holding the support plate P/P′ against the adjacent vertebrae V₁ and V₂ while the distal ends of the threaded fasteners 10 and 20 are embedded in the bone. The threaded fasteners 10 and 20 thus converge from their proximal ends towards their distal ends with the first threaded fastener 10 defining at its distal end a female threaded opening 12 that extends obliquely with respect to a longitudinal orientation of the first threaded fastener 10. This threaded opening 12 is adapted to be threadably engaged by the male threaded distal end of the second fastener 20. The proximal end of the first fastener is provided with indicia so as to indicate an orientation of the threaded opening 12 within the vertebrae V₁/V₂ thereby facilitating the engagement therein of the male threaded distal end of the second fastener 20. With the support plate P/P′, the first and second fasteners 10 and 20 define a triangular frame that is firmly secured to the vertebrae V₁ and V₂.

It is an object of the present invention to provide a novel anchoring system for securing various objects to bones, such as spinal devices or instrumentations to the rachis and plates or other to broken bones.

It is also an object of the present invention to provide an anchoring system well adapted to prevent loosening thereof over time.

### SUMMARY OF THE INVENTION

According to one aspect of the invention, an anchoring system for bones is provided, wherein the anchoring system comprising male and female anchoring members each having proximal and distal ends, said proximal ends being adapted, when installed, to be spaced from each other with said male and female anchoring members converging from said proximal ends towards said distal ends, said male and female anchoring members being adapted to be connected to each other in the bone and distally of said proximal ends via a connection mechanism, said male anchoring member being provided with a thread proximally of said connection mechanism, said thread being adapted to substantially securely engaging bone material, whereby said male and female anchoring members are connected together by said connection mechanism thereby securing said male and female anchoring members to the bone, with said anchoring system being further anchored to the bone by said thread.

In accordance with another aspect of the invention, a dual threaded fastener is provided, comprising at least first and second sections provided with first and second threads respectively, said first section defining a chamber with a proximal head of said second section being longitudinally slidable within said chamber, said first and second sections being adapted to be rotatable independent of one another and being telescopically mounted together, said first and second threads being of different configuration for firm engagement into different media.

In accordance with yet another aspect of the invention, a method of installing an object to a bone is provided, comprising the steps of: (a) providing first and second anchoring members, said first anchoring member including first and second sections provided respectively with a thread and a distal end, said female anchoring member having a distal end; (b) inserting said first and second anchoring members in the bone while engaging the object and with said thread of said first member engaging bone material; and (c) handling said second section of said first anchoring member for securing said distal ends of said first and second anchoring members together, while said first section of said male anchoring member remains stationary.

### BRIEF DESCRIPTION OF THE DRAWINGS

Having thus generally described the nature of the invention, reference will now be made to the accompanying drawings, showing by way of illustration a preferred embodiment thereof, and in which:

FIG. 1 is a schematic cross-sectional plan view of a bridging plate mounted to a lumbar vertebra using an anchoring system of the prior art;

FIG. 2 is a schematic anterior perspective view of a bridging plate mounted to a pair of cervical vertebra using the prior art anchoring system of FIG. 1;

FIG. 3 is a schematic perspective view of an anchoring system in accordance with the present invention;

FIG. 4 is a perspective view of a female pin of the anchoring system of FIG. 3;

FIG. 5 is a schematic perspective view, partly in cross section, of a male anchor of the anchoring system of FIG. 3;

FIG. 6 is a perspective view of an inner member of the male anchor of FIG. 5;

FIG. 7 is a perspective view of an outer member of the male anchor of FIG. 5;

FIG. 8 is a schematic perspective view of the male anchor in a partly retracted position;

FIG. 9 is a perspective view similar to FIG. 8 but showing the male anchor in an extended position thereof;

FIG. 10 is a vertical cross-sectional view of the male anchor of FIG. 9; and

FIG. 11 is a schematic top plan view of part of an upper part of the outer member of the male anchor of FIG. 10.

### DESCRIPTION OF THE PREFERRED EMBODIMENTS

The present invention relates to a new anchoring system including a new male anchor that includes at least two different threads adapted for different receiving materials or media, wherein the different threads can be independently screwed into the materials. To do so, there is provided a differential-type connection between the two threads and typically the two threads are coaxial and are capable of relative telescopic axial movement therebetween and in which each segment of this telescope can be screwed independently from the other.

For the purpose of understanding the principles of the invention, reference will now be made to the embodiments illustrated in the drawings.

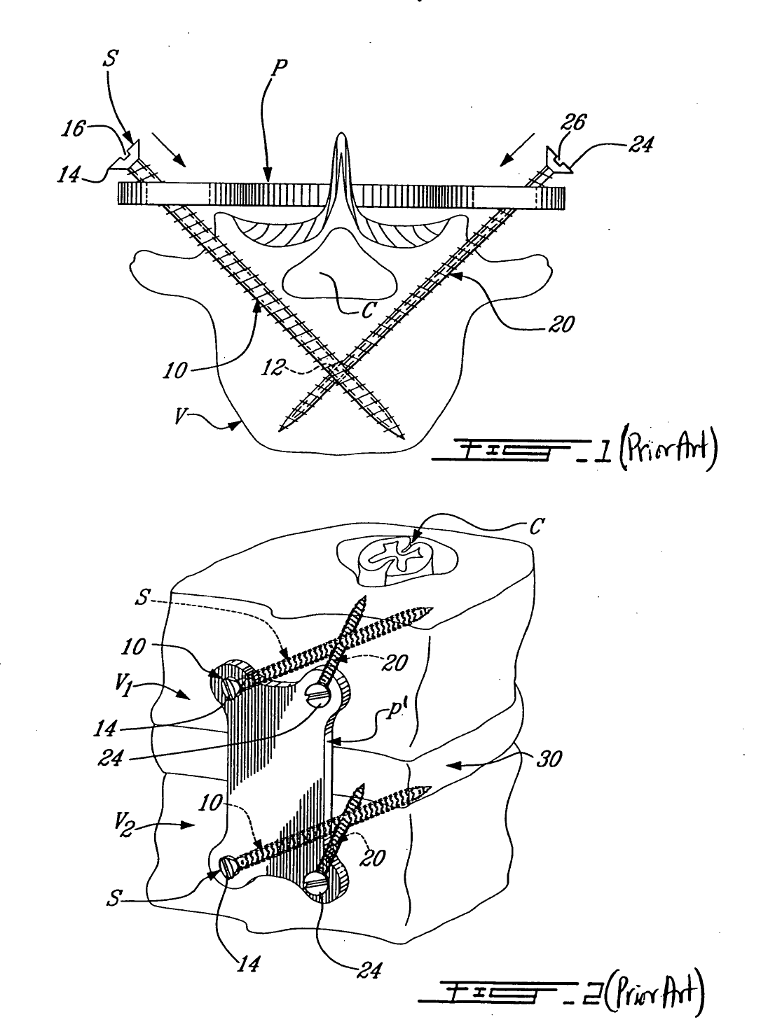

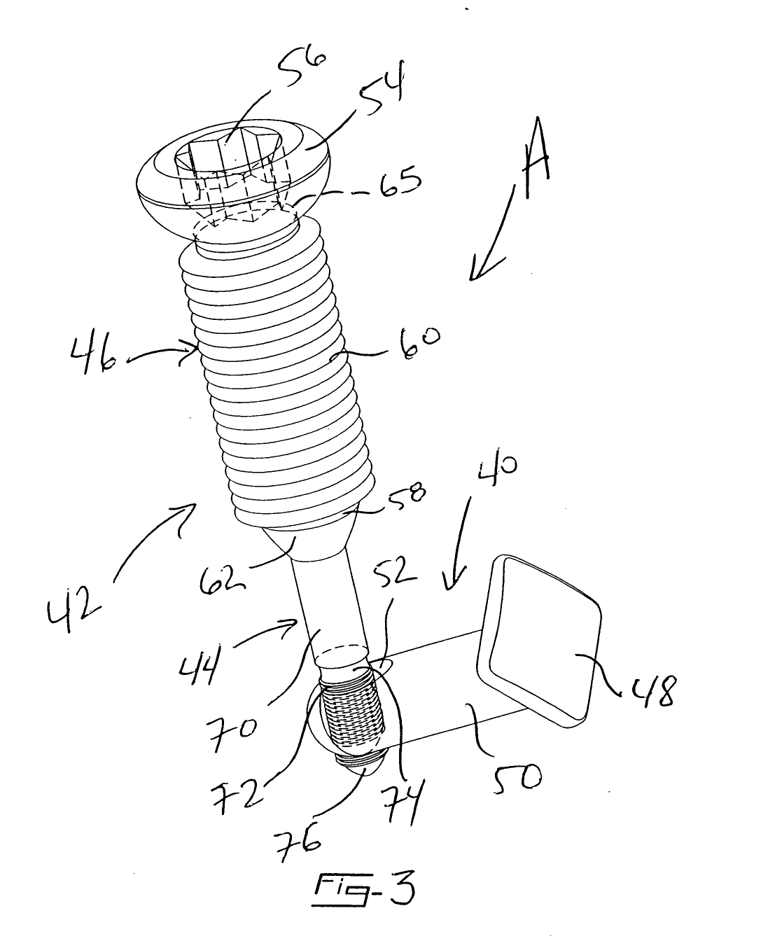

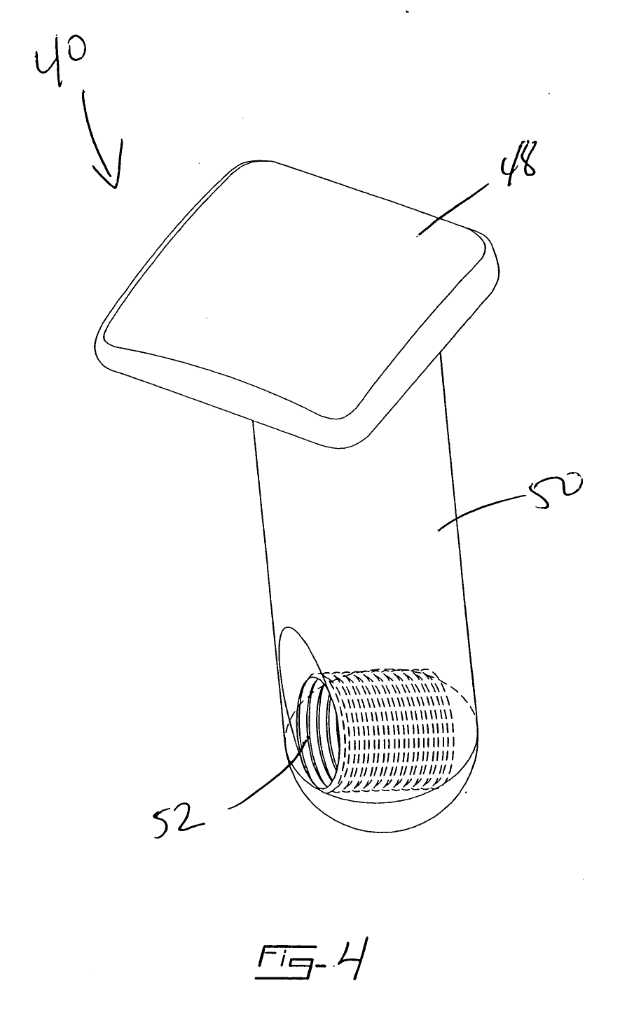

FIG. 3 illustrates an anchoring system A in accordance with one aspect of the invention. When desired, the anchoring system A could be used with a support plate, such as support plates P and P′ shown in FIGS. 1 and 2 in order to connect to adjacent vertebrae together or to any object to be fixed to the bones. In such a case, the anchoring system A would replace the threaded fasteners 10 and 20 of FIGS. 1 and 2.

As shown in FIG. 3, the anchoring system A includes a female pin 40 and a male anchor 42. The female pin 40 includes a proximal head 48 of a non-circular shape and an oblique opening 52. The male anchor 42 includes an inner member 44 and an outer member 46, wherein the inner member 44 is being adapted to be slidable along the outer member 46, and the inner member 44 is rotatable with respect to the outer member 46.

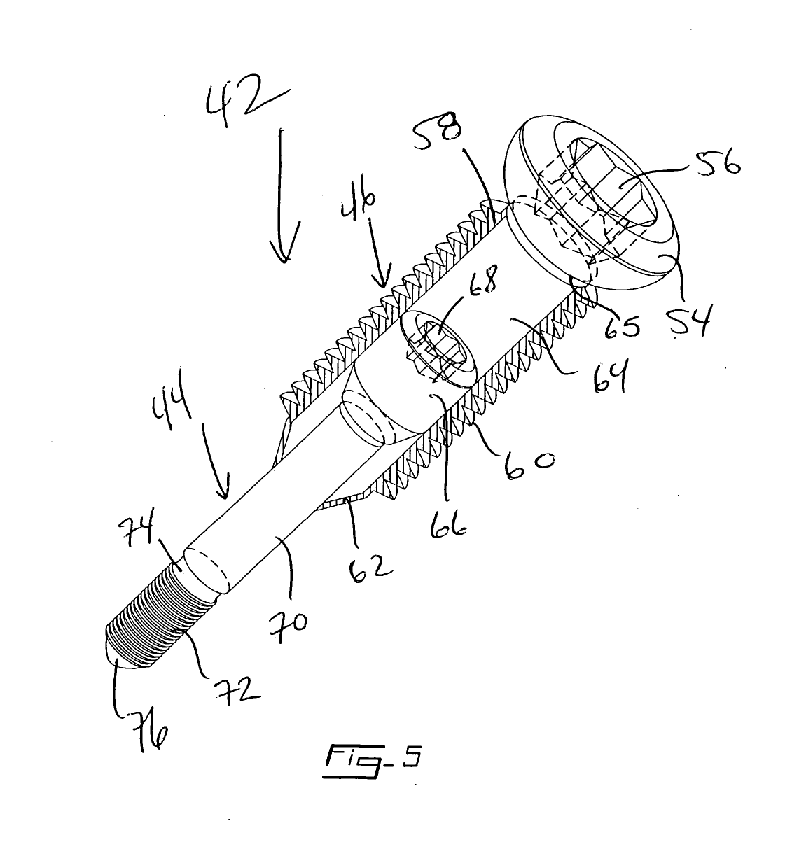

In accordance with one embodiment of the invention, as shown in FIG. 4, the female pin 40 includes a proximal head 48 of a non-circular shape and adapted to lodge in a corresponding recess defined in the object to be fixed. This feature is intended to position the female threaded connection in the right direction to receive the male member according to a predetermined angulation.

The proximal head 48 may take on different shapes, such as square, rectangular, polygonal, oval, etc., as long as it is not circular thereby ensuring that the female pin 40 can only take on one position with respect to the object. This is further ensured in the present embodiment by the fact that a shank 50 of the female pin 40 is mounted in an offset way to the head 48.

The shank 50 of the female pin 40 has a smooth outer surface and defines at a distal end thereof an oblique opening 52 that is tapped such that it can be engaged by the male threads of a threaded fastener.

In the present embodiment, the opening 52 is adapted to threadably receive a distal thread of the male anchor 42, as it will be explained in details hereinafter. The proximal head 48 and the threaded opening 52 are defined one with respect to the other along unique angulation in the three spatial planes (axial rotation, in the sagittal plane, inclination in the frontal and horizontal planes).

The noncircular design of the head 48 allows it to be received in a stereo-specific manner in the recess defined in the object to be fixed and hence determines an ideal positioning of its distal end of the threaded opening 52. This pre-positioning provides the necessary precision required for subsequently threadably engaging the male anchor 42 into the female pin 40 along this predefined angle.

As the shank 50 is smooth, the female pin 40 is generally inserted in a translational manner although it may be somewhat rotated along a longitudinal axis of the shank 50 such as to properly position the head 48 in the aforementioned recess.

It should be noted that when desired, the proximal head 48 of the female pin 40 could also be circular, as long as it is provided with a slot therein for receiving a screwing tool, and as long as it is provided with an indicia that indicates the direction of the distal threaded opening 52 in order to ensure a proper positioning thereof prior to engagement of the male anchor 42 therein.

In accordance with another embodiment of the invention, the distal opening 52 does not have to be threaded. In such a case, the distal opening has a configuration that allows the male anchor to be secured to the female pin, for instance, by way of a clipping mechanism or by a locking mechanism resulting from a partial rotation (e.g. ¼ turn) of the male anchor with respect to the female pin once the male anchor is engaged in the distal opening of the female pin. In other words, the male anchor can translationally slide through the distal opening of the female pin when the male anchor has a given orientation but becomes locked to the female pin after the male anchor has been rotated to a second, i.e. locking, orientation.

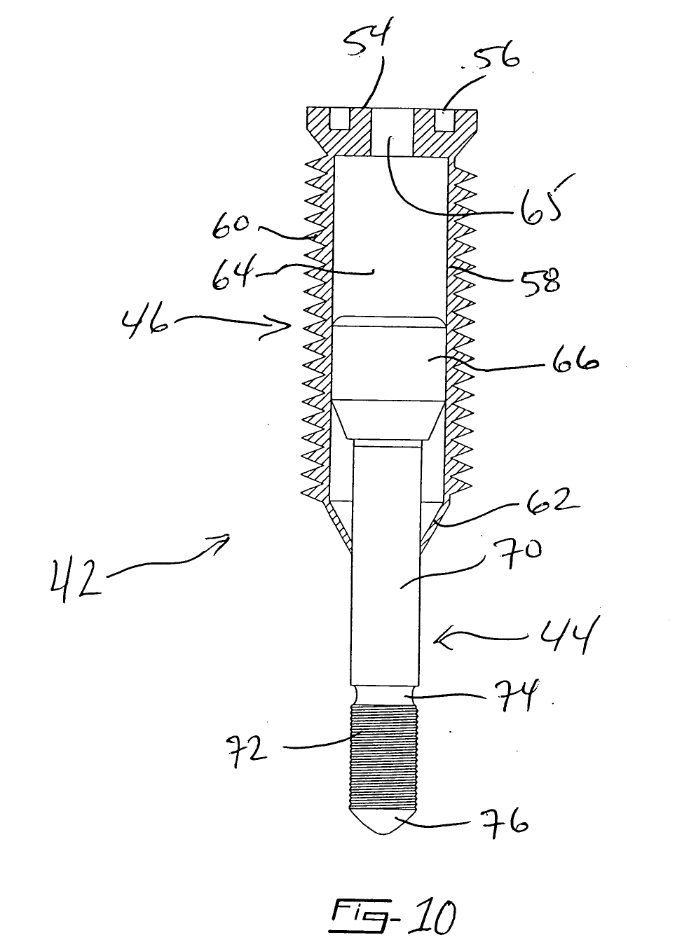

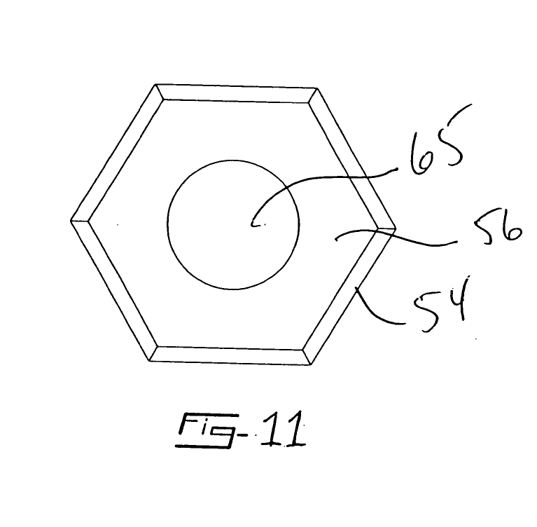

Referring now to FIGS. 5 and 7, the outer member 46 of the male anchor 42 includes a proximal head 54 defining a socket 56 adapted to be engaged by a torque-producing tool for rotation of the outer member 46. The outer member 46 also includes a hollow shank 58 provided with a large thread 60 therearound. Distally of the hollow shank 58, there is provided a frusto-conical tip 62. The hollow shank 58 and the tip 62 define a chamber 64 that is long enough to accommodate, for instance completely, therein the inner member 44. An opening 65 (also shown in FIGS. 10 and 11) defined between the socket 56 and the chamber 64. The large thread 60 is deep and self-tapping such as to firmly engage bone tissues.

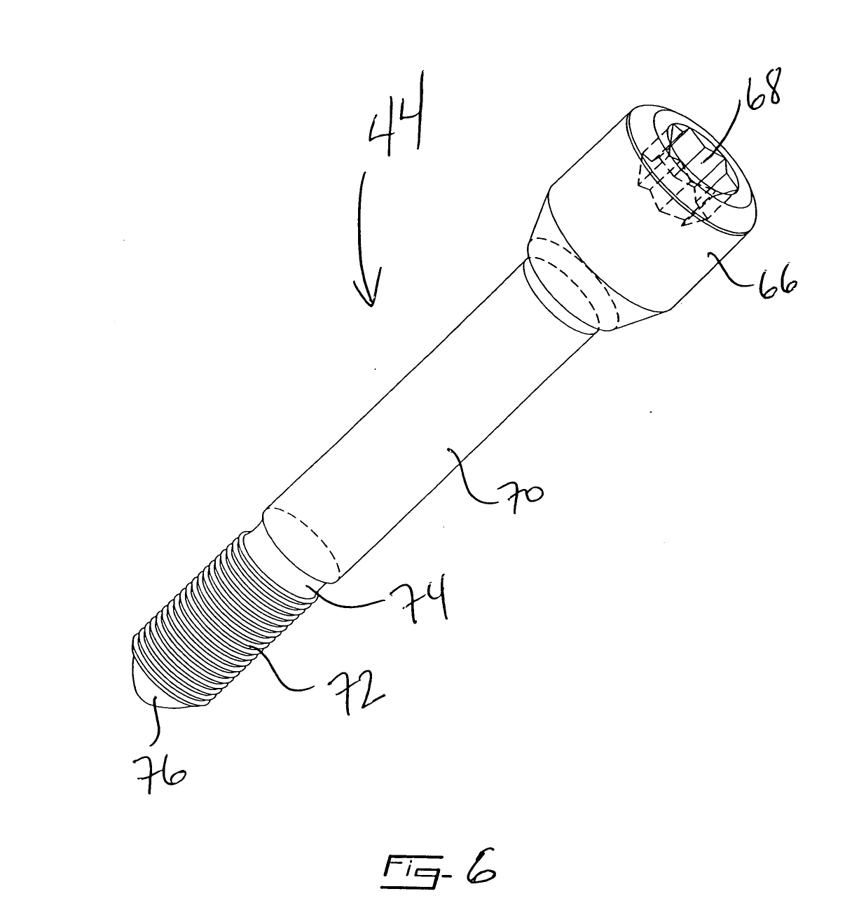

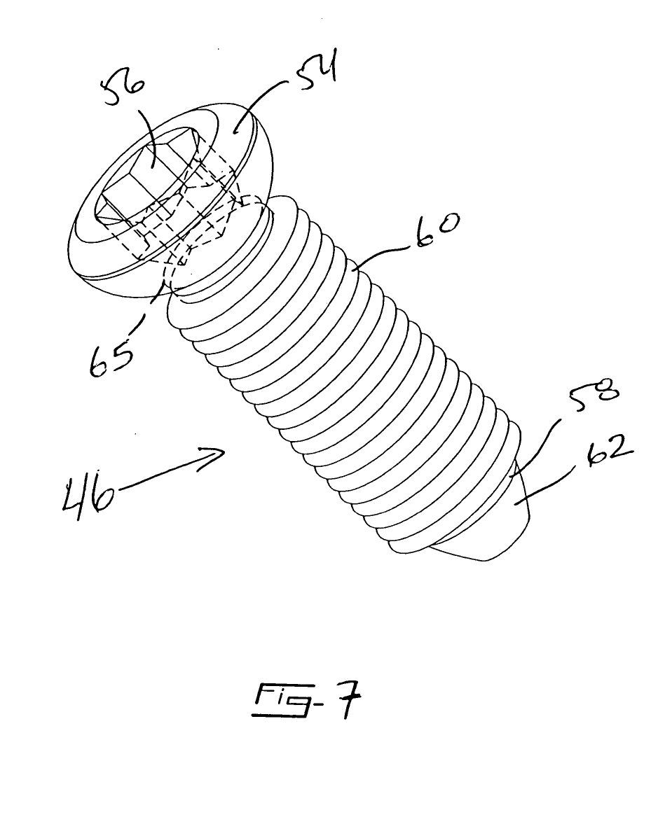

Referring now to FIG. 6, the inner member 44 comprises a proximal head 66 that defines a socket 68 adapted to be engaged by a torque-producing tool. The inner member 44 also includes a smooth shank 70 and a threaded distal end 72. A peripheral arcuate groove 74 is provided between the shank 70 and the threaded distal end 72. The thread of the distal end 72 is adapted to threadably engage the tapped opening 52 of the female pin 40 (shown in FIG. 3).

As best seen in FIG. 5, the proximal head 66 of the inner member 44 is adapted to slidably displace longitudinally within the chamber 64 defined in the outer member 46. The tapered tip 62 of the outer member 46 and the proximal head 66 of the inner member 44 co-act to prevent the inner member 44 from sliding out of the outer member 46. Accordingly, the inner member 44 can displace, with respect to the outer member 46, translationally between retracted and extended positions thereof.

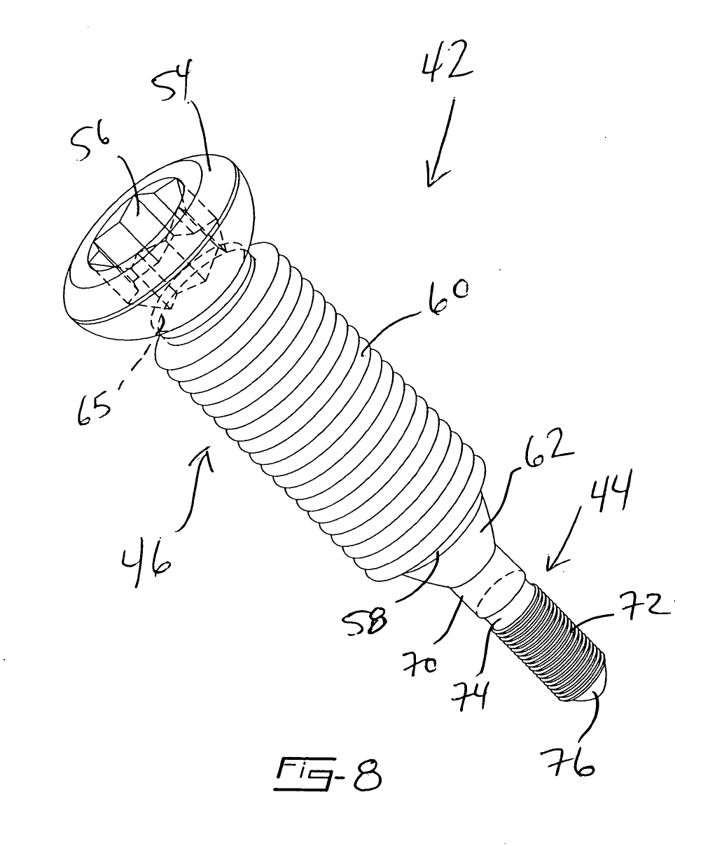

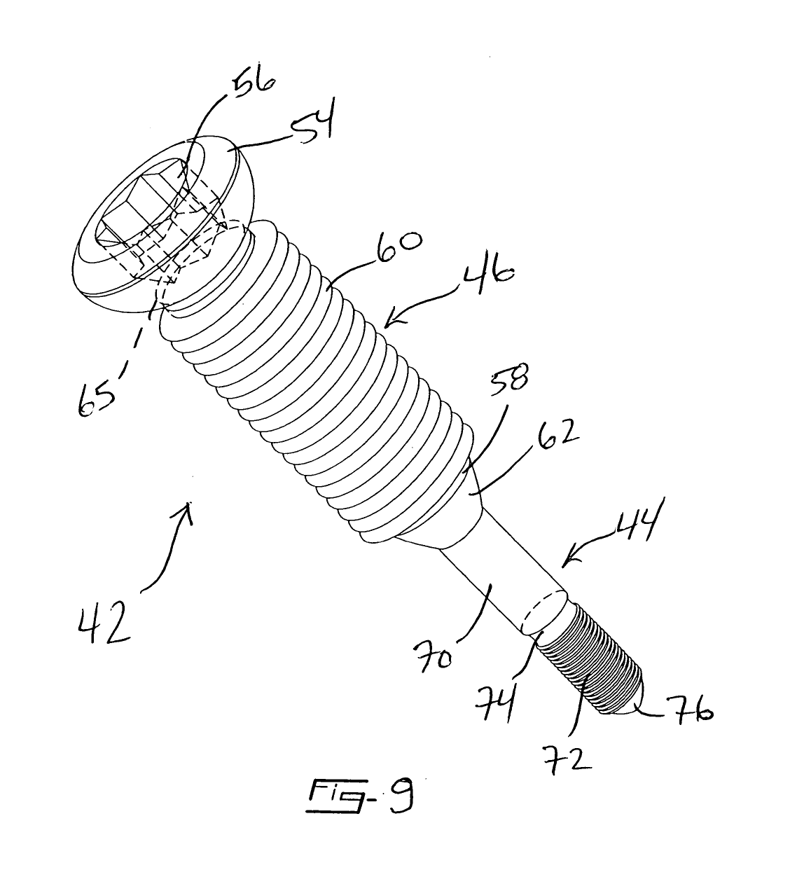

FIG. 8 illustrates the inner member 44 respectively in a partly retracted position and FIG. 9 illustrates the inner member 44 in a completely extended position.

The opening 65 of the outer member 46 is larger than the tool that will be used to rotate the inner member 44 to allow for this tool to be inserted in the chamber 64 and to be engaged to the proximal head 66 of the inner member 44. Also, the opening 65 of the outer member 46 is smaller than the tool that will be used to rotate the outer member 46 to provide an abutment for this tool below the socket 56 when it is desired to engage this tool to the head 54 of the outer member 46 via the socket 56 thereof for screwing the outer member 46 into, for instance, bone tissue.

The inner member 44, asides from being slidable along the outer member 46, is rotatable with respect thereto such that the inner and outer members 44 and 46 can be rotated independently from one another.

The head 66 of the inner member 44 is high enough, i.e. in the longitudinal orientation of the inner member 44, to ensure proper guidance to the sliding motion thereof within the chamber 64 of the outer member 46.

In a fully retracted position of the inner member 44 with respect to the outer member 46, the proximal head 64 of the inner member 44 abuts the distal part of the head 54 of the outer member 46, which defines the opening 65, with the threaded distal end 72 of the inner member 44 extending within the tapered tip 62 of the outer member 46, only an unthreaded tip 76 of the inner member 44 extending outwardly of the outer member 46.

This position of the male anchor 42 results in a substantially conventional overall screw configuration for allowing the initial engagement of the male anchor 42 in the bone, during which the outer member 46 engages the bone tissues while the threaded distal end 72 of the inner member 44 is inactive. Once the outer member 46 is firmly anchored in the bone tissues and the female pin has been properly positioned in the bone tissues, the inner member 44 is rotated, using a tool engaged in the socket 68, until the distal threaded end 72 thereof has engaged the tapped opening 52 of the female pin. The inner member 44 is so rotated until the anchoring system A is a rigid structure engaged in the bone tissues.

In the fully extended position of the inner member 44 with respect to the outer member 46, the proximal head 64 of the inner member 44 abuts the tapered tip 62 of the outer member 46, with the shank 70, the threaded distal end 72 and the groove 74 of the inner member 44 extending outwardly of the outer member 46. More particularly, the lower part of the head 66 is tapered to corresponding with the inner surface of the tapered tip 62 of the outer member 46 for proper abutment therebetween in the fully extended position.

The smooth shank 70 of the inner member 44 can vary in length from zero (where it is non existent) to the length of the chamber 64 such that, in the fully extended position, substantially only the threaded distal end 72 and the tip 76 of the inner member 44 extend out of the outer member 46.

The threaded distal end 72 of the inner member 44 has a thread that is different than the thread 60 of the outer member 46 and is typically a machine screw-type thread (thin and of a small pitch) for threadably engaging the tapped opening 52 of the female pin 40. The length of the threaded distal end 72 can vary such that it can extend right up to the head 66, in which case there is no smooth shank 70. The ratio of the length of the chamber 64 of the outer member 46 on the length of the threaded distal end 72 can vary from 1 to 100%.

Other connection mechanisms than threads can be used to engage the distal end 72 of the inner member 44 of the male anchor 42 with the opening 52 of the female pin 40 does not have to be threads. Clips and locking mechanisms, as mentioned hereinabove, and other means can be used to secure the male anchor 42 to the female pin 40.

Both sockets 56 and 68 can take various configurations (e.g. rectangular slot, polygonal recess, cruciform grooves, etc.) and sizes.

It is possible for the inner member 44, in its retracted position, to be completely encased in the chamber 64 of the outer member 46. Alternatively, the threaded distal end 72 and the shank 70 can be of lengths such that the former and even the latter can be visible when the inner member 44 is in its extended position, and this configuration can be useful when the female pin 40 to be engaged is distanced from the tip 62 of the outer member 46 of the male anchor 42 or when a material/tissue of a different density is interposed, for instance, between the bone and the female pin 40.

The male anchor 42 can include more than two telescopic members, with each member having a thread adapted to firmly engage a given medium.

A compressed spring (not shown) may be provided in the chamber 64 so as to extend therein between the head 54 of the outer member 46 and the head 66 of the inner member 44 for acting on the head 66 of the inner member 44 and bias the latter towards the extended position. A passageway is defined centrally of the spring to allow a tool to engage and rotate the inner member 44. Such a spring facilitates the screwing of the outer member 46 by offering to the material/bone tissue the pointed tip 76 of the inner member 44 instead of the dull frusto-conical tip 62 of the outer member 46. The spring also assists the screwing process of the inner member 44 as, by maintaining a constant pressure thereon, the operator only has to rotate the tool that is without having to exert axial pressure thereon, thereby making the process safer.

The advantage of the present male anchor 42, which has multiple differently threaded sections adapted to engage different materials/tissues in an adequate if not optimal way and which is adapted to the physico-chemical characteristics of the media traversed thereby, is to allow the outer member 46 of the male anchor 42 to become anchored in the bone via a self-tapping thread, and to thus provide more long-term stability and solidity by favouring a fusion with the bone.

It is contemplated to use the male anchor 42 without the female pin 40, that is, for instance, by providing a thread on the distal end 72 of the inner member 44, which is adapted (instead of engaging the tapped opening 52 of the female pin 40) to engage a material/tissue different than that engaged by the thread 60 of the outer member 46.

It will be readily apparent to those skilled in the art that various changes and modifications of an obvious nature may be made, and all such changes and modifications are considered to fall within the scope of the appended claims. Other embodiments of the invention will be apparent to those skilled in the art from consideration of the specification and practice of the invention disclosed herein. It is intended that the specification and examples be considered as exemplary only, with a true scope and spirit of the invention being indicated by the following claims and their equivalents.

1. An anchoring system for bones, comprising male and female anchoring members each having proximal and distal ends, said proximal ends being adapted, when installed, to be spaced from each other with said male and female anchoring members converging from said proximal ends towards said distal ends, said male and female anchoring members being adapted to be connected to each other in the bone and distally of said proximal ends via a connection mechanism, said male anchoring member being provided with a thread proximally of said connection mechanism, said thread being adapted to substantially securely engaging bone material, whereby said male and female anchoring members are connected together by said connection mechanism thereby securing said male and female anchoring members to the bone, with said anchoring system being further anchored to the bone by said thread.

2. An anchoring system as defined in claim 1, wherein said connection mechanism comprises a male thread and a female tap provided on one and the other of said male and female anchoring members and adapted for threaded engagement, said thread of said male anchoring member being of different configuration than said male thread of said connection mechanism.

3. An anchoring system as defined in claim 2, wherein said male thread is provided at said distal end of said male anchoring member, said female tap being provided in an opening defined obliquely in said distal end of said female anchoring member.

4. An anchoring system as defined in claim 1, wherein said female anchoring member comprises a pin provided at said proximal end with a head.

5. An anchoring system as defined in claim 4, in combination with an object adapted to be mounted exteriorly of the bone, said anchoring system being adapted to extend through the object and into the bone with said proximal ends of said male and female anchoring members being adapted to secure the object to the bone.

6. The combination of the anchoring system and the object of claim 5, wherein the object is a support plate.

7. The combination of the anchoring system and the object of claim 5, wherein said head is adapted to engage a recess defined in the object such that said female anchoring member can take a single orientation with respect to the object thereby facilitating the identification of a position of said distal end of said female anchoring member in the bone and a subsequent engagement of said male anchoring member to said female anchoring member via said connection mechanism.

8. An anchoring system as defined in claim 1, wherein said male anchoring member comprises at least first and second sections provided respectively with said thread and with at least part of said connection mechanism, said first and second sections being rotatable independently of one another.

9. An anchoring system as defined in claim 8, wherein said first and second sections are telescopically mounted together, said first section defining a chamber with a proximal head of said second section being longitudinally slidable within said chamber.

10. An anchoring system as defined in claim 9, wherein a distal end of said first section prevents said proximal head of said second section from sliding out of said chamber.

11. An anchoring system as defined in claim 10, wherein said distal end of said first section includes a frusto-conical tip with a shank of said second section being slidable therein, said frusto-conical tip providing an abutment for said proximal head of said second section in an extended-most position of said second section relative to said first section.

12. An anchoring system as defined in claim 9, wherein said proximal end of said male anchoring member defines an opening for allowing a first tool to be inserted in said chamber and to engage said proximal head of said second section.

13. An anchoring system as defined in claim 12, wherein said proximal end of said male anchoring member defines an abutment for allowing a second tool to engage said proximal head of said male anchoring member for acting on said second section.

14. An anchoring system as defined in claim 12, wherein a spring is provided in said chamber for acting on said proximal head of said second section such as to bias said second section towards an extended position thereof, said spring defining a passageway for allowing the first tool to engage said proximal head of said second section.

15. An anchoring system as defined in claim 1, wherein said proximal end of said female anchoring member includes an indicia for indicating a relative position of said distal end of said female anchoring member.

16. A dual threaded fastener, comprising at least first and second sections provided with first and second threads respectively, said first section defining a chamber with a proximal head of said second section being longitudinally slidable within said chamber, said first and second sections being rotatable independently of one another and being telescopically mounted together, said first and second threads being of different configuration for firm engagement into different media.

17. A dual threaded fastener as defined in claim 16, wherein a distal end of said first section prevents said proximal head of said second section from sliding out of said chamber.

18. A dual threaded fastener as defined in claim 17, wherein said distal end of said first section includes a frusto-conical tip with a shank of said second section being slidable therein, said frusto-conical tip providing an abutment for said proximal head of said second section in an extended-most position of said second section relative to said first section.

19. A dual threaded fastener as defined in claim 16, wherein a proximal end of said first section defines an opening for allowing a first tool to be inserted in said chamber and to engage said proximal head of said second section.

20. A dual threaded fastener as defined in claim 19, wherein said proximal end of said first section defines an abutment for allowing a second tool to engage said proximal head of said first section for acting on said second section.

21. A dual threaded fastener as defined in claim 19, wherein a spring is provided in said chamber for acting on said proximal head of said second section such as to bias said second section towards an extended position thereof, said spring defining a passageway for allowing the first tool to engage said proximal head of said second section.

22. A method of installing an object to a bone, comprising the steps of:
(a) providing first and second anchoring members, said first anchoring member including first and second sections provided respectively with a thread and a distal end, said female anchoring member having a distal end; (b) inserting said first and second anchoring members in the bone while engaging the object and with said thread of said first member engaging bone material; and
(c) handling said second section of said first anchoring member for securing said distal ends of said first and second anchoring members together, while said first section of said male anchoring member remains stationary.
